(19) United States
(12) Patent Application Publication
(10) Pub. No.: US 2006/0183219 A1
(43) Pub. Date: Aug. 17, 2006

(54) Patterning substrate and cell culture substrate

(75) Inventors: Hideyuki Miyake, Tokyo (JP); Hideshi Hattori, Tokyo (JP); Hironori Kobayashi, Tokyo (JP)

Correspondence Address: SEYFARTH SHAW LLP, 55 E. MONROE STREET, SUITE 4200, CHICAGO, IL, 60603-5803, US

(73) Assignee: Dai Nippon Printing Co., Ltd.

(21) Appl. No.: 11/044,124

(22) Filed: Jan. 27, 2005

(30) Foreign Application Priority Data
Jan. 28, 2004 (JP) 2004-020214

(51) Int. Cl. B05D 3/00; G01N 1/31; C12M 3/00

(52) U.S. Cl. 435/289.1; 427/2.13; 427/553

(57) ABSTRACT

The present invention intends primarily to provide such as a cell culture patterning substrate that is used to adhere cells in a highly precise pattern on a base material to culture and a cell culture substrate on which cells are adhered in a highly precise pattern. To attain the object, the invention provides a patterning substrate comprising: a base material; and a cell adhesion-inhibiting layer which is formed on the base material and comprises a cell adhesion-inhibiting material that has cell adhesion-inhibiting properties of inhibiting adhesion to cells and is denatured by action of a photocatalyst on the basis of irradiation with energy.

### BACKGROUND OF THE INVENTION

1. Field of the Invention

The present invention relates to a cell culture patterning substrate capable of adhering cells in a highly precise pattern, a patterning substrate used for forming the cell culture patterning substrate, a coating liquid for patterning substrate used for forming the patterning substrate, and a cell culture substrate on which cells are adhered in a highly precise pattern.

2. Description of the Related Art

At present, cell cultures of various animals and plants are performed, and also new cell culture methods are in development. The technologies of the cell culture are utilized, such as, to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culturing plate with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method of arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in Japanese Patent Application Laid-Open (JP-A) No. 2-245181, an electric charge-retaining medium on which an electrostatic pattern is formed is applied to culture cells for the purpose of proliferating nerve cells in a form of circuit, and the like. Furthermore, JP-A No. 3-7576 tries to arrange cultured cells on a surface on which a cell adhesion-inhibiting or cell adhesive photosensitive hydrophilic polymer has been patterned by a photolithography method.

Furthermore, JP-A No. 5-176753 discloses a cell culture base material on which a substance such as collagen and the like affecting on the adhesion ratio and form of cells is patterned, and a method of producing this base material by a photolithography method. By culturing cells on such a base material, a larger amount of cells can be adhered on a surface on which collagen or the like is patterned, to realize patterning of cells.

However, such patterning of cell culture regions may be required to be highly precise depending on applications. In the case of conducting patterning by a photolithography method using a photosensitive material as described above, a highly precise pattern can be obtained; however, a cell adhesive material is required to have photosensitivity, and it is difficult in many cases to conduct chemical modification to impart such photosensitivity to, for instance, biopolymers and the like; thereby leading to extremely narrow width in selectivity of cell adhesive materials, problematically. Furthermore, in a photolithography method using a photo resist, it is necessary to use a liquid developer and the like, and these can affect adversely in culturing cells in some cases.

Furthermore, as a method of forming a highly precise pattern of a cell adhesive material, a Micro Contact Printing method is proposed by George M. Whitesides, Harvard University (for example, U.S. Pat. Nos. 5,512,131 and 5,900,160, JP-A Nos. 9-240125 and 10-12545 etc). However, there is a problem in that it is difficult to industrially produce a cell culture base material having a pattern of a cell adhesive material using this method.

### SUMMARY OF THE INVENTION

In this connection, it is desired to provide such as a cell culture patterning substrate that is used to cause cells to adhere in highly precise pattern onto a base material to culture, and a cell culture substrate to which cells are adhered in highly precise pattern.

The present invention provides a patterning substrate comprising: a base material; and a cell adhesion-inhibiting layer which is formed on the base material and comprises a cell adhesion-inhibiting material that has cell adhesion-inhibiting properties of inhibiting adhesion to cells and is denatured by action of a photocatalyst on a basis of irradiation with energy.

According to the invention, the cell adhesion-inhibiting layer comprises the cell adhesion-inhibiting material; therefore, the cell adhesive properties of the cell adhesion-inhibiting layer can be made low. In the case of using, for example, a photocatalyst-containing layer comprising a photocatalyst to irradiate energy onto this cell adhesion-inhibiting layer, the cell adhesion-inhibiting material is denatured to render the layer a layer having cell adhesive properties. Accordingly, the present patterning substrate can be rendered a patterning substrate wherein a region having cell adhesive properties and a region low in cell adhesive properties can easily be formed by irradiation with energy in a pattern form, using, for example, the above-mentioned photocatalyst-containing layer, and the like.

The present invention also provides a patterning substrate comprising: a base material; and a cell adhesion-inhibiting layer which is formed on the base material and comprises a binder and a cell adhesion-inhibiting material that has cell adhesion-inhibiting properties of inhibiting adhesion to cells and is decomposed or denatured by action of a photocatalyst on the basis of irradiation with energy.

According to the invention, the cell adhesion-inhibiting layer comprises the cell adhesion-inhibiting material; therefore, the cell adhesive properties of the cell adhesion-inhibiting layer can be made low. In the case of using, for example, a photocatalyst-containing layer comprising a photocatalyst to irradiate energy onto this cell adhesion-inhibiting layer, the cell adhesion-inhibiting material is decomposed or denatured to render the region where the binder is exposed, a region which comprises the binder and a decomposition product of the cell adhesion-inhibiting material, or some other region that has cell adhesive properties. Accordingly, the present patterning substrate can be rendered a patterning substrate wherein a region having cell adhesive properties and a region low in cell adhesive properties can easily be formed by irradiation with energy in a pattern form, using, for example, the above-mentioned photocatalyst-containing layer, and the like.

In the invention, it is preferred that the cell adhesion-inhibiting layer comprises a cell adhesive material having cell adhesive properties at least after the material is irradiated with the energy. This makes it possible to render the above-mentioned patterning substrate a patterning substrate wherein the region irradiated with the energy has better cell adhesive properties.

In the invention, it is preferred that a light-shielding portion is formed on the base material. This makes it possible to render the above-mentioned patterning substrate a patterning substrate wherein the cell adhesion-inhibiting material only inside the region where no light-shielding portion is formed can be decomposed or denatured by arranging the cell adhesion-inhibiting layer and, for example, a photocatalyst-containing layer comprising a photocatalyst so as to be facing each other and then irradiating energy onto the entire surface of the resultant from the base material side thereof.

The present invention also provides a cell culture patterning substrate, wherein the cell adhesion-inhibiting layer of the above-mentioned patterning substrate has a cell adhesion portion wherein the cell adhesion-inhibiting material is decomposed or denatured in a pattern form and a cell adhesion-inhibiting portion which is a region other than the above-mentioned cell adhesion portion.

According to the invention, the cell culture patterning substrate comprises the cell adhesion portion having cell adhesive properties, where the cell adhesion-inhibiting material is decomposed or denatured, and the cell adhesion-inhibiting portion low in cell adhesive properties, where the cell adhesion-inhibiting material is not decomposed or denatured; therefore, the cell culture patterning substrate can be rendered a cell culture patterning substrate capable of causing cells to adhere highly precisely only onto the cell adhesion portion without requiring any complicated step, any treating solution or the like that produces a bad effect on the cells.

The present invention also provides a cell culture substrate, wherein the cells adhere onto the cell adhesion portion of the above-mentioned cell culture patterning substrate.

According to the invention, on the cell culture patterning substrate, the cell adhesion portion having cell adhesive properties and the cell adhesion-inhibiting portion low in cell adhesive properties are formed; therefore, cells can be caused to adhere easily only onto the cell adhesion portion so as to produce a cell culture substrate onto which the cells adhere in a highly precise pattern form. In this case, the cell culture substrate does not need to contain therein any photocatalyst; accordingly, the culture substrate also has an advantage that there is no possibility that the cells are affected by any photocatalyst with the passage of time.

The present invention also provides a coating liquid for patterning substrate comprising a cell adhesion-inhibiting material which has cell adhesion-inhibiting properties of inhibiting adhesion to cells and is decomposed or denatured by action of a photocatalyst on a basis of irradiation with energy, and a cell adhesive material which has cell adhesive properties at least after the material is irradiated with the energy.

According to the invention, by applying the coating liquid for patterning substrate onto, for example, a base material and then using such as a photocatalyst-containing layer comprising a photocatalyst to irradiate energy onto the resultant, the cell adhesion-inhibiting material can easily be decomposed so that the region irradiated with the energy can be rendered a region having cell adhesive properties; and further this region irradiated with the energy comprises the cell adhesive material. For these reasons, the region can be rendered a region better in cell adhesive properties. On the other hand, the region not irradiated with the energy inhibits cells from adhering onto this region through the cell adhesion-inhibiting material; therefore, the region can be rendered a region low in cell adhesive properties. Accordingly, the coating liquid for patterning substrate can be rendered a coating liquid for patterning substrate capable of forming easily a region good in cell adhesive properties and a region low in cell adhesive properties.

The present invention also provides a method for producing a cell culture patterning substrate, comprising: a cell adhesion-inhibiting layer forming process of forming, on a base material, a cell adhesion-inhibiting layer comprising a cell adhesion-inhibiting material that has cell adhesion-inhibiting properties of inhibiting adhesion to cells and is decomposed or denatured by action of a photocatalyst on the basis of irradiation with energy; and

an energy irradiating process of arranging the cell adhesion-inhibiting layer and a photocatalyst-containing layer side substrate having a photocatalyst-containing layer comprising the photocatalyst and a base body to dispose the cell adhesion-inhibiting layer and the photocatalyst-containing layer facing each other, and then irradiating the energy onto a resultant from a given direction to form a pattern composed of a cell adhesion portion wherein the cell adhesion-inhibiting material comprised in the cell adhesion-inhibiting layer is decomposed or denatured, and a cell adhesion-inhibiting portion which is other than the cell adhesion portion.

According to the invention, the cell adhesion-inhibiting layer formed through the cell adhesion-inhibiting layer forming process comprises the cell adhesion-inhibiting material; therefore, by using the photocatalyst-containing layer side substrate to irradiate energy in a pattern form in the energy irradiating process, it is possible to produce a cell culture patterning substrate wherein the following portions are easily formed: a cell adhesion portion having cell adhesive properties, where the cell adhesion-inhibiting material is decomposed or denatured, and a cell adhesion-inhibiting portion low in cell adhesive properties, which is not irradiated with the energy. According to the invention, the cell culture patterning substrate does not need to comprise therein any photocatalyst since the above-mentioned photocatalyst-containing layer side substrate is used. It is therefore possible to produce a high-quality cell culture patterning substrate wherein even if, for example, cells adhere onto the cell adhesion portion, the cells are not affected by any photocatalyst with the passage of time.

The invention also provides a method for producing a cell culture substrate, comprising a cell adhesion process of causing the cells to adhere onto the cell adhesion portion of the cell culture patterning substrate produced by the above-mentioned method for producing the cell culture patterning substrate.

According to the invention, the cell adhesion portion having cell adhesive properties and the cell adhesion-inhibiting portion low in cell adhesive properties are formed on the cell culture patterning substrate; it is therefore possible to easily produce a cell culture substrate wherein cells adhere highly precisely only onto the cell adhesion portion.

### BRIEF DESCRIPTION OF THE DRAWINGS

FIG. 1 is a schematic sectional view showing an example of the patterning substrate of the present invention.

FIG. 2 is a schematic sectional view showing another example of the patterning substrate of the invention.

FIG. 3 is a schematic sectional view showing an example of the cell culture patterning substrate of the invention.

FIG. 4 is a schematic sectional view showing another example of the cell culture patterning substrate of the invention.

FIGS. 5A to 5C are process charts showing an example of a method for forming cell adhesion-inhibiting portions in the cell culture patterning substrate of the invention.

FIG. 6 is a schematic sectional view showing an example of the photocatalyst-containing layer side substrate used in the invention.

FIG. 7 is a schematic sectional view showing another example of the photocatalyst-containing layer side substrate used in the invention.

FIG. 8 is a schematic sectional view showing still another example of the photocatalyst-containing layer side substrate used in the invention.

FIG. 9 is a schematic sectional view showing an example of the cell culture substrate of the invention.

FIGS. 10A and 10B are each a schematic sectional view for explaining a base material used in the cell culture patterning substrate of the invention.

### DESCRIPTION OF THE PREFERRED EMBODIMENT

The present invention relates to such as a cell culture patterning substrate on which cells can be adhered in highly precise pattern, a patterning substrate that is used to form the cell culture patterning substrate, a coating liquid for patterning substrate that is used to form the patterning substrate, and a cell culture substrate on which cells are adhered in highly precise pattern. Hereinafter, these will be explained below separately.

A. Coating Liquid for Patterning Substrate

First, the coating liquid for patterning substrate of the present invention is described. The coating liquid for patterning substrate is a coating liquid comprising: a cell adhesion-inhibiting material that has cell adhesion-inhibiting properties of inhibiting adhesion to cells and is decomposed or denatured by action of a photocatalyst on the basis of irradiation with energy; and a cell adhesive material that has cell adhesive properties at least after the material is irradiated with the energy.

The coating liquid for patterning substrate of the invention comprises the cell adhesion-inhibiting material having the cell adhesion-inhibiting properties; therefore, the layer obtained by applying the coating liquid for patterning substrate onto a base material or the like can be rendered a layer low in cell adhesive properties. By using such as a photocatalyst-containing layer comprising a photocatalyst to irradiate energy onto the obtained layer, the cell adhesion-inhibiting material is decomposed or denatured so that the cell adhesive properties are not inhibited; and further the layer comprises the cell adhesive material. For these reasons, the cell adhesive properties of the layer can be made good. Accordingly, a region good in cell adhesive properties and a region low in cell adhesive properties can easily be formed without using any complicated step or any treating solution or the like that produces a bad effect on cells.

The following will describe each of the constituents used in the above-mentioned coating liquid for patterning substrate.

1. Cell Adhesion-Inhibiting Material

A cell adhesion-inhibiting material that is used in the coating liquid for patterning substrate according to the present invention will be explained. The cell adhesion-inhibiting material used in the coating liquid for patterning substrate according to the invention, as far as it has cell adhesion-inhibiting properties that inhibit cells from adhering and can be decomposed or denatured by action of the photocatalyst on the basis of irradiation with energy, is not particularly restricted in the kind and the like.

Here, the wording “has the cell adhesion-inhibiting properties” means to have a property that inhibits cells from adhering to the cell adhesion-inhibiting material, and, when cell adhesive properties is different depending on the kind of cells and the like, means to have the property of inhibiting from adhering with a target cell.

As the cell adhesion-inhibiting material used in the present invention, one that has such cell adhesion-inhibiting properties and can be decomposed or denatured by action of the photocatalyst on the basis of irradiation with energy to lose the cell adhesion-inhibiting properties or to become good in the cell adhesive properties can be used.

As the cell adhesion-inhibiting material, materials high in, for instance, the hydration ability can be used. When the material high in the hydration ability is used, water molecules gather in the vicinity of the cell adhesion-inhibiting material to form a hydrated layer. Normally, such material high in the hydration ability is higher in adhesive properties with water molecules than that with the cell; accordingly, the cells cannot adhere to the material high in the hydration ability, resulting in one low in the cell adhesive properties. Here, “the hydration ability” means a property of hydrolyzing with water molecules, and “the hydration ability being high” means that it can readily hydrolyze with water molecules.

As materials that are high in the hydration ability and can be used as the cell adhesion-inhibiting material, for instance, polyethylene glycol, amphoteric ion materials having a betaine structure or the like, and phosphatide-containing materials and the like can be cited. In the case of such a material being used as the cell adhesion-inhibiting material, when energy is irradiated, by action of the photocatalyst, the cell adhesion-inhibiting material is decomposed, modified or the like, the hydrated layer on a surface comes off, and thereby the cell adhesion-inhibiting material is rendered one that does not have the cell adhesion-inhibiting properties.

A surfactant having a water repellent or oil repellent organic group that can be decomposed by action of the photocatalyst also can be used. As such surfactant, hydrocarbons base surfactants such as the respective series of NIKKO L, BL, BC, BO, and BB manufactured by Nikko Chemicals Co., Ltd., and fluorine base or silicone base nonionic surfactants such as ZONYL FSN and FSO manufacture by Du Pont Kabushiki Kaisya, Surflon S-141 and 145 manufactured by ASAHI GLASS CO., LTD., Megaface F-141 and 144 manufactured by DAINIPPON INK AND CHEMICALS, Inc., FTERGENT F-200 and F251 manufactured by NEOS, UNIDYNE DS-401 and 402 manufactured by DAIKIN INDUSTRIES, Ltd., and Fluorad FC-170 and 176 manufactured by 3M can be cited, and furthermore cationic surfactants, anionic surfactants and amphoteric surfactants also can be used.

In the case of such a material being use as the cell adhesion-inhibiting material, when the coating liquid for patterning substrate is coated to form a layer, the cell adhesion-inhibiting material is distributed unevenly on a surface. Thereby, the water repellency or oil repellency of the surface of the cell adhesion-inhibiting layer can be made higher; accordingly, one small in the interaction with cells and low in the cell adhesive properties can be obtained. Furthermore, when energy is irradiated on the layer, by action of the photocatalyst, the cell adhesion-inhibiting material is readily decomposed to expose the photocatalyst, resulting in one that does not have the cell adhesion-inhibiting properties.

In the invention, it is particularly preferred to use, as the cell adhesion-inhibiting material, a material the cell adhesive properties of which become good by action of a photocatalyst on the basis of irradiation with energy. Such a cell adhesion-inhibiting material is preferably a material which has oil repellency or water repellency and has a surface changed so as to have an appropriate hydrophilicity or lipophilicity by action of a photocatalyst on the basis of irradiation with energy, thereby exhibiting good cell adhesive properties.

In the case of using, as the cell adhesion-inhibiting material, the above-mentioned material having water repellency or oil repellency, interactions, such as hydrophobic interaction, between cells and the cell adhesion-inhibiting material can be made small by the water repellency or oil repellency of the cell adhesion-inhibiting material. As a result, the cell adhesive properties of the material can be made low. When the surface of the material is changed so as to have an appropriate water repellency or oil repellency, the cell adhesive properties of the material can be made large.

As a material having such water repellency or the oil repellency, for instance, one where a skeleton has such a high bond energy that cannot be decomposed by action of the photocatalyst and that has a water repellent or oil repellent organic substitution group that can be decomposed by action of the photocatalyst can be cited.

As one that has the skeleton having such high bond energy that cannot be decomposed by action of the photocatalyst and the water repellent or oil repellent organic substitution group that can be decomposed by action of the photocatalyst, for instance, (1) organopolysiloxane that exhibits large strength by hydrolyzing or polycondensating chloro, alkoxysilane or the like owing to a sol-gel reaction and the like, and (2) organopolysiloxane or the like in which reactive silicones are crosslinked can be cited.

In the case of the (1), it is preferable to be organopolysiloxane that is a hydrolysis condensate or cohydrolysis condensate of at least one kind of silicon compounds expressed by a general formula:
YₙSiX₍₄₋ₙ₎
(Here, Y denotes an alkyl group, fluoroalkyl group, vinyl group, amino group, phenyl group or epoxy group, or organic groups including these, and X denotes an alkoxyl group, acetyl group or halogen. n is an integer of 0 to 3). The number of carbons of the group expressed with Y is preferably in the range of 1 to 20, and the alkoxy group expressed with X is preferably a methoxy group, ethoxy group, propoxy group or butoxy group.

As the organic groups, in particular, polysiloxane containing a fluoroalkyl group can be preferably used. Specifically, a hydrolysis condensate or cohydrolysis condensate of at least one kind of fluoroalkylsilanes below can be cited. Ones generally known as the fluorinated silane coupling agents can be used.
CF₃(CF₂)₃CH₂CH₂Si(OCH₃)₃;
CF₃ (CF₂)₅CH₂CH₂Si (OCH₃)₃;
CF₃ (CF₂)₇CH₂CH₂Si (OCH₃)₃;
CF₃ (CF₂)₉CH₂CH₂Si (OCH₃)₃;
(CF₃)₂CF(CF₂)₄CH₂CH₂Si (OCH₃)₃;
(CF₃)₂CF(CF₂)₆CH₂CH₂Si (OCH₃)₃;
(CF₃)₂CF(CF₂)₈CH₂CH₂Si (OCH₃)₃;
CF₃ (C₆H₄)C₂H₄Si (OCH₃)₃;
CF₃ (CF₂)₃(C₆H₄)C₂H₄Si (OCH₃)₃;
CF₃ (CF₂)₅(C₆H₄)C₂H₄Si (OCH₃)₃;
CF₃ (CF₂) 7 (C₆H₄)C₂H₄Si (OCH₃)₃;
CF₃ (CF₂)₃CH₂CH₂SiCH₃ (OCH₃)₂;
CF₃ (CF₂)₅CH₂CH₂SiCH₃ (OCH₃)₂;
CF₃ (CF₂)₇CH₂CH₂SiCH₃ (OCH₃)₂;
CF₃ (CF₂)₉CH₂CH₂SiCH₃ (OCH₃) 2;
(CF₃)₂CF (CF₂)₄CH₂CH₂SiCH₃ (OCH₃) 2;
(CF₃)₂CF (CF₂)₆CH₂CH₂Si CH₃ (OCH₃) 2;
(CF₃)₂CF(CF₂)₈CH₂CH₂Si CH₃(OCH₃)₂;
CF₃ (C₆H₄)C₂H₄SiCH₃ (OCH₃) 2;
CF₃ (CF₂) 3 (C₆H₄)C₂H₄SiCH₃ (OCH₃) 2;
CF₃ (CF₂) 5 (C₆H₄)C₂H₄SiCH₃ (OCH₃)₂;
CF₃ (CF₂) 7 (C₆H₄)C₂H₄SiCH₃ (OCH₃)₂;
CF₃ (CF₂)₃CH₂CH₂Si (OCH₂CH₃) 3;
CF₃ (CF₂)₅CH₂CH₂Si (OCH₂CH₃) 3;
CF₃ (CF₂)₇CH₂CH₂Si (OCH₂CH₃) 3;
CF₃ (CF₂)₉CH₂CH₂Si (OCH₂CH₃)₃;
CF₃ (CF₂)₇SO₂N(C₂H₅)C₂H₄CH₂Si (OCH₃)₃

When polysiloxane containing such a fluoroalkyl group is used as the cell adhesion-inhibiting material, one high in the water repellency or oil repellency can be formed, resulting in rendering one small in the interaction with cells and low in the cell adhesive properties. Furthermore, when energy is irradiated on such a material, readily, fluorine and the like can be removed and an OH group and the like can be introduced on a surface to render the interaction with cells larger; accordingly, the cell adhesive properties can be made good.

As the reactive silicone according to the (2), compounds having a skeleton expressed by a general formula below can be cited.

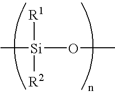

In the above general formula, n denotes an integer of 2 or more, R¹ and R² each represents a substituted or nonsubstituted alkyl group, alkenyl group, aryl group or cyanoalkyl group having 1 to 20 carbons, and a vinyl, phenyl and halogenated phenyl occupy 40% or less by mole ratio to a total mole. Furthermore, one in which R¹ and R² each is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain.

Together with the organopolysiloxane, a stable organosilicium compound that does not cause a crosslinking reaction such as dimethylpolysiloxane may be blended separately.

When such reactive silicone is used, one high in the water repellency or oil repellency can be obtained; that is, one small in the interaction with cells and low in the cell adhesive properties can be obtained. Furthermore, when energy is irradiated on such a material, readily, a substituent group can be removed and an OH group and the like can be introduced on a surface to render the interaction with cells larger; accordingly, the cell adhesive properties can be made good.

In the case of using an organopolysiloxane or reactive silicone as described above, the compound is denatured by action of the photocatalyst on the basis of irradiation with energy. However, the case gives an advantage that the compound can fulfill a part of a binder also in the layer obtained by applying the coating liquid for patterning substrate since the skeleton thereof remains without being decomposed.

In the case of using the material having water repellency or oil repellency described-above as the cell adhesion-inhibiting material, the material is usually a material the contact angle of which with water is preferably 80° or more, more preferably from 100 to 130°. According to this, the cell adhesive properties thereof can be made low. The upper limit of the angle is the upper limit of the contact angle of the cell adhesion-inhibiting material with water on a flat base material. In the case of measuring the contact angle of the cell adhesion-inhibiting material with water on, for example, a base material having irregularities, the upper limit may be about 160° as described in data, for example, Ogawa et al., Japanese Journal of Applied Physics, vols. 2 and 32, L614-L615 (1993).

When the cell adhesion-inhibiting material is irradiated with energy so as to be rendered a material having cell adhesive properties, it is preferred to irradiate the energy to set the contact angle with water into the range of 10 to 40°, in particular 15 to 30° C. This makes it possible to render the cell adhesion-inhibiting material a material high in cell adhesive properties.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

The content by percentage of such cell adhesion-inhibiting material in the coating liquid for patterning substrate is preferably from 0.001 to 60% by weight, more preferably from 0.01 to 40% by weight, and even more preferably from 0.1 to 20% by weight. This makes it possible to render the region comprising the cell adhesion-inhibiting material a region low in cell adhesive properties.

When energy is irradiated on the cell adhesion-inhibiting material such as mentioned above to decompose or denature and thereby to use as one good in the cell adhesive properties, the energy may be irradiated to decompose or denature the cell adhesion-inhibiting material to an extent that makes the adhesive properties with target cell good, and there is no need of completely decomposing or denaturing the cell adhesion-inhibiting material.

The cell adhesion-inhibiting material is preferably a material having surface activity for the following reason: in the step of applying and drying the coating liquid for patterning substrate, the ratio of the material distributed unevenly in the surface of the coating film becomes high to give good cell adhesion-inhibiting properties.

2. Cell Adhesive Material

In the next place, a cell adhesive material used in the coating liquid for patterning substrate according to the present invention will be explained. A cell adhesive material used in the present invention, as far as it has the cell adhesive properties at least after energy is irradiated, is not particularly restricted. It may be one that is used as a binder or one that is used separately from the binder. Furthermore, for instance, it may be one that has the good cell adhesive properties prior to energy irradiation or one that becomes good in cell adhesive properties by action of the photocatalyst on the basis of irradiation with energy. Here, “having the cell adhesive properties” means being good to adhere with cells and, when the cell adhesive properties is different depending on kinds and the like of cells, means being good to adhere with a target cell.

In the present invention, as far as the cell adhesive material has good cell adhesive properties at least after energy is irradiated, it may be one of which cell adhesive properties is made good owing to physical interactions such as an hydrophobic interaction, electrostatic interaction, hydrogen bonding, van der Waals force and the like or owing to biological characteristics.

As a material that has the cell adhesive properties owing to the physical interactions, specifically, temperature sensitive polymers such as hydrophilic polystyrene and poly (N-isopropylacrylamide), basic polymers such as polylysine, basic compounds such as aminopropyltriethoxysilane, N-(2-aminoethyl)-3-aminopropyltrimethoxysilane and condensates or the like including these can be cited.

As a material having the cell adhesive properties owing to the biological characteristics, specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like can be cited.

In the invention, the amount of the cell adhesive material is appropriately optimized in accordance with the cell adhering ability of the material. Usually, the content by percentage thereof in the coating liquid for patterning substrate is preferably from 0.0001 to 30% by weight, more preferably from 0.001 to 10% by weight. This makes it possible that at the time of applying the coating liquid for patterning substrate to form a layer, the cell adhesive properties of the region irradiated with energy are made better. In the case of using, as the cell adhesive material, the material having good cell adhesive properties before the irradiation thereof with energy, it is preferred that the material is contained to such a degree that the cell adhesion-inhibiting properties of the cell adhesion-inhibiting material are not inhibited in the region not irradiated with energy after the coating liquid for patterning substrate is applied to form a layer.

3. Coating Liquid for Patterning Substrate

The following will describe the coating liquid for patterning substrate of the present invention. The coating liquid for patterning substrate is not limited to any special kind if the coating liquid comprises the above-mentioned cell adhesive material and cell adhesion-inhibiting material. If necessary, the coating liquid may appropriately comprise other components such as a binder. The incorporation of the binder makes it possible to make the application of the coating liquid onto, for example, a base material easy and gives various properties such as strength and resistance to the formed layer. The binder is appropriately selected in accordance with the use purpose of the coating liquid for patterning substrate. In the invention, the part of this binder may be fulfilled by the cell adhesion-inhibiting material or the cell adhesive material.

It is preferred that the coating liquid for patterning substrate of the invention does not contain any photocatalyst. This makes the following possible: for example, in the case of causing cells to adhere onto the layer formed by the application of the coating liquid for patterning substrate, the resultant member is rendered a member wherein no photocatalyst produces an effect on the cells with the passage of time.

B. Patterning Substrate

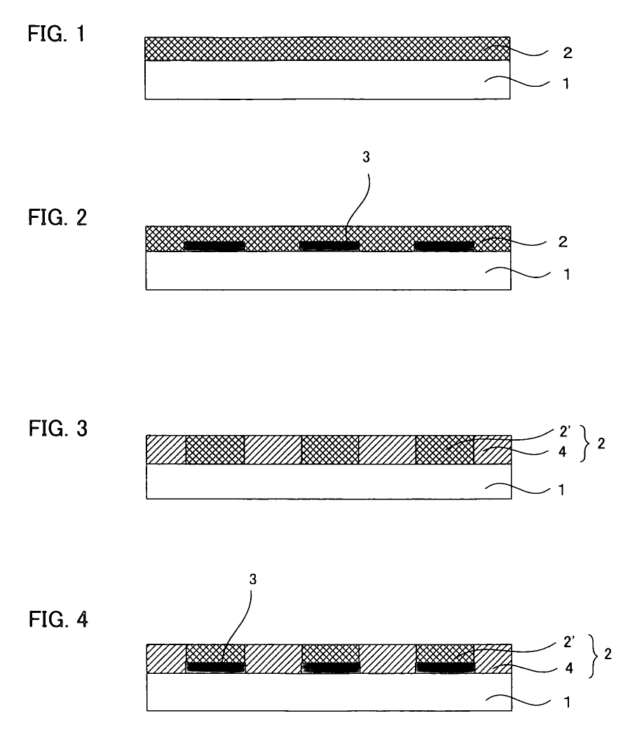

The following will describe the patterning substrate of the present invention. The patterning substrate is classified into two embodiments depending on the structure of its cell adhesion-inhibiting layer. As shown in, for example, FIG. 1, any one of the embodiments has a base material 1 and a cell adhesion-inhibiting layer 2 which is formed on the base material 1, has cell adhesion-inhibiting properties, and is denatured and the like by action of a photocatalyst on the basis of irradiation with energy. The following will describe each of the embodiments in detail.

### 1. FIRST EMBODIMENT

First, the first embodiment of the patterning substrate of the invention is described. The patterning substrate is a member comprising: a base material; and a cell adhesion-inhibiting layer which is formed on the base material and comprises a cell adhesion-inhibiting material that has cell adhesion-inhibiting properties of inhibiting adhesion to cells and is denatured by action of a photocatalyst on the basis of irradiation with energy.

In the present embodiment, the cell adhesion-inhibiting material is a material which is denatured by action of a photocatalyst on the basis of irradiation with energy; therefore, for example, by using such as a photocatalyst-containing layer comprising the photocatalyst to irradiate energy onto the cell adhesion-inhibiting layer, the cell adhesion-inhibiting material is denatured so that the cell adhesion-inhibiting properties thereof decrease. As a result, a region having cell adhesive properties can be formed. On the other hand, in the region not irradiated with the energy, the cell adhesive properties thereof can be made low by the cell adhesion-inhibiting properties of the cell adhesion-inhibiting material. Therefore, according to the present embodiment, it is possible to provide a patterning substrate capable of forming easily a region having cell adhesive properties, where the cell adhesion-inhibiting material is denatured, and a region having poor cell adhesive properties, where the cell adhesion-inhibiting material is not changed, by irradiation with energy by use of, for example, the photocatalyst-containing layer.

As shown in, for example, FIG. 2, the patterning substrate of the present embodiment may be a member where light-shielding portions 3 are formed on a base material 1 and a cell adhesion-inhibiting layer 2 is formed on the light-shielding portions 3. This makes it possible that in the case of arranging the above-mentioned photocatalyst-containing layer or the like and the cell adhesion-inhibiting layer to be facing each other and subsequently irradiating energy onto the resultant from the base material side thereof, the photocatalyst in the photocatalyst-containing layer inside the region where the light-shielding portions are formed is not excited so that the cell adhesion-inhibiting material only in the region where the light-shielding portions are not formed can be denatured. Thus, this patterning substrate has an advantage that highly precise cell adhesion portions can easily be formed without using the positioning of a photomask or other operations.

The following will describe each of the constituents of the patterning substrate of the present embodiment.

(Cell Adhesion-Inhibiting Layer)

First, the cell adhesion-inhibiting layer used in the present embodiment is described. The cell adhesion-inhibiting layer is a layer which is formed on the base material which will be detailed later, and comprises a cell adhesion-inhibiting material that has cell adhesion-inhibiting properties of inhibiting adhesion onto cells and is denatured by action of a photocatalyst on the basis of irradiation with energy.

The cell adhesion-inhibiting layer in the embodiment is not limited to any special kind if the layer is a layer comprising the cell adhesion-inhibiting material. For example, when the cell adhesion-inhibiting material fulfills a part of a binder also, the layer may not contain any other binder or the like. In the case of using such as a photocatalyst-containing layer to irradiate energy onto the cell adhesion-inhibiting layer, the cell adhesion-inhibiting material is denatured in the region irradiated with the energy; consequently, the cell adhesion-inhibiting properties of the region decrease so that the region becomes a region having cell adhesive properties. As the cell adhesion-inhibiting material, there can be used a material which is denatured by action of a photocatalyst on the basis of irradiation with energy, such as organopolysiloxane or reactive silicone, out of the cell adhesion-inhibiting materials described in the item “A. Coating Liquid for Patterning Substrate”.

In the embodiment, it is particularly preferred that the cell adhesion-inhibiting layer comprises therein a cell adhesive material. This makes it possible that when the cell adhesion-inhibiting layer is irradiated with energy, the cell adhesive properties of the region irradiated with the energy are made better so as to cause cells to adhere, in a more highly precise pattern, only onto the region irradiated with the energy.

In the embodiment, the cell adhesion-inhibiting layer can be formed, for example, by applying such as a coating liquid comprising the above-mentioned cell adhesion-inhibiting material. In the case of incorporating the cell adhesive material also into the layer as described above, the layer can be formed by using a coating liquid for patterning substrate which comprises the cell adhesive material besides the cell adhesion-inhibiting material.

The method for forming the cell adhesion-inhibiting layer may be a method of applying the coating liquid for patterning substrate by an ordinary coating method. Specific examples of the coating method include such as spin coating, spray coating, dip coating, roll coating, and bead coating. A method for forming a super thin film, such as an adsorption process or alternate adsorption process, can also be preferably used.

When concave portions are formed in the base material which will be detailed later, it is possible to use: a casting process of dropping down the coating liquid for patterning substrate into the concave portions in the base material, and drying the liquid so as to form the cell adhesion-inhibiting layer; an adsorption process of dropping down such as the coating liquid for patterning substrate into the concave portions in the base material, and washing the surface after a given time; or some other process.

The cell adhesion-inhibiting material, the cell adhesive material and others that are used in the cell adhesion-inhibiting layer in the embodiment are equivalent to those described in the item “A. Coating Liquid for Patterning Substrate”. Thus, description thereof is omitted herein.

The film thickness of the cell adhesion-inhibiting layer is appropriately selected in accordance with the kind of the patterning substrate and so on, and is usually from about 0.001 to 1.0 μm, preferably from about 0.01 to 0.3 μm.

It is preferred that the cell adhesion-inhibiting layer in the embodiment does not contain any photocatalyst. This makes it possible that, for example, when cells are caused to adhere onto the cell adhesion-inhibiting layer, the cells are prevented from being affected by any photocatalyst with the passage of time. Thus, the patterning substrate can be rendered a high-quality patterning substrate. The method for decomposing or denaturing the cell adhesion-inhibiting material in the cell adhesion-inhibiting layer may be a method, for example, of disposing a photocatalyst-containing layer comprising a photocatalyst to face to the cell adhesion-inhibition layer and then irradiating energy onto the resultant, as described above.

(Base Material)

The following will describe the base material used in the present embodiment. The base material is not limited to any special kind if the base material is a material on which the cell adhesion-inhibiting layer can be formed. Examples thereof include inorganic materials such as metal, glass and silicon, and organic materials such as plastic.

The flexibility and the like of the base material are properly selected according to the kind of the patterning substrate, applications, or the like. Furthermore, the transparency of the base material is properly selected depending on such as the kind of the patterning substrate, or a direction in which energy that is irradiated to decompose or denature the cell adhesion-inhibiting material is irradiated. For instance, when the base material has such as the light-shielding portion and the energy is irradiated from a base material side and the like, the base material has the transparency.

In the embodiment, the base material may be a flat base material or a base material wherein one or more concave portions are formed. The base material may be a base material wherein a single concave portion is formed as shown in FIG. 10A, or a base material wherein plural concave portions are formed as shown in FIG. 10B.

At this time, side walls of the base material having the concave portion(s) may be treated in such a manner that the cell adhesion-inhibiting layer will not be formed thereon. Examples of the method for such a treatment include a method of using a mask or the like to cause a material having liquid repellency to adhere only onto the side walls by CVD; and a method of causing a material having liquid repellency to adhere onto the entire surface of the concave portion(s) and then using a cylindrical mask or the like to conduct ultraviolet ray treatment, plasma treatment or some other treatment, thereby making only the bottom face(s) of the concave portion(s) lyophilic.

In the embodiment, the base material may be washed with a medical liquid, such as an alkali solution, and may be subjected to dry washing, such as oxygen plasma treatment or ultraviolet treatment. In this case, the wettability of the coating liquid for patterning substrate or the like is improved. Furthermore, this case has an advantage that the adhesive property of the cell adhesion-inhibiting layer is improved since reactive functional groups are arranged on the surface of the base material.

On the base material according to the present embodiment, as mentioned above, a light-shielding portion may be formed. The light-shielding portion that can be used in the embodiment, as far as it can shield energy that is irradiated on the patterning substrate, is not particularly restricted. For instance, a metal thin film that is made of chromium or the like and formed into a thickness of about 1000 to 2000 Å by a sputtering method, a vacuum deposition method or the like is formed and patterned to form a shielding portion. As the patterning method, an ordinary patterning method such as the sputtering can be used.

A method may be one by which a layer that contains light-shielding particles such as carbon particulates, metal oxides, inorganic pigments and organic pigments in a resin binder is formed in a pattern. As the resin binders that can be used, a polyimide resin, acrylic resin, epoxy resin, polyacrylamide, polyvinyl alcohol, gelatin, casein, cellulose and the like can be used singularly or in combination of two or more kinds, and furthermore a photosensitive resin and an O/W emulsion type resin composition such as emulsified reactive silicone can be used A thickness of such resinous light-shielding portion can be set in the range of 0.5 to 10 μm. As a method of patterning such resinous light-shielding portion, methods such as a photolithography method and a printing method that are generally used can be used.

The light-shielding portions may be formed on the base material surface of the side at which the cell adhesion-inhibiting layer is formed, or may be formed on the opposite surface.

(Patterning Substrate)

The following will describe the patterning substrate of the present embodiment. The patterning substrate is not limited to any special kind if the patterning substrate is a member wherein the cell adhesion-inhibiting layer is formed on the base material. If necessary, for example, one or more different layers may be laminated thereon.

### 2. SECOND EMBODIMENT

The following will describe the second embodiment of the patterning substrate of the present invention. The patterning substrate of the embodiment is a member comprising: a base material; and a cell adhesion-inhibiting layer which is formed on the base material and comprises a binder and a cell adhesion-inhibiting material that has cell adhesion-inhibiting properties of inhibiting adhesion to cells and is decomposed or denatured by action of a photocatalyst on the basis of irradiation with energy.

According to the present embodiment, the cell adhesion-inhibiting material is decomposed or denatured by action of a photocatalyst on the basis of irradiation with energy; therefore, for example, by using such as a photocatalyst-containing layer comprising the photocatalyst to irradiate energy onto the cell adhesion-inhibiting layer, the cell adhesion-inhibiting material is decomposed or denatured so that the cell adhesion-inhibiting properties of the region irradiated with the energy are lowered, whereby a region having cell adhesive properties can be formed. On the other hand, in the region not irradiated with the energy, the cell adhesive properties thereof can be made low through the cell adhesion-inhibiting properties of the cell adhesion-inhibiting material. Therefore, according to the embodiment, it is possible to produce a patterning substrate capable of forming easily a region having cell adhesive properties, where the cell adhesion-inhibiting material is denatured, and a region poor in cell adhesive properties, where the cell adhesion-inhibiting material is not changed, by irradiation with energy using, for example, the photocatalyst-containing layer.

The patterning substrate of the embodiment may be, for example, a patterning substrate wherein light-shielding portions are formed on a base material and a cell adhesion-inhibiting layer is formed on the light-shielding portions in the same manner as in the first embodiment. The base material, the light-shielding portions and others that are used in the embodiment are equivalent to those used in the first embodiment. Thus, description thereof is omitted herein. The following will describe the cell adhesion-inhibiting layer used in the patterning substrate of the embodiment.

(Cell Adhesion-Inhibiting Layer)

The cell adhesion-inhibiting layer in the embodiment is not limited to any special kind if the layer is a layer comprising the above-mentioned cell adhesion-inhibiting material and binder. The cell adhesion-inhibiting material used in the embodiment is decomposed or denatured by action of a photocatalyst on the basis of irradiation with energy; therefore, in the case of using such as a photocatalyst-containing layer to irradiate energy onto the cell adhesion-inhibiting layer, the cell adhesion-inhibiting material is decomposed or denatured in the region irradiated with the energy so that the cell adhesion-inhibiting properties thereof are lowered. As a result, the region becomes a region having cell adhesive properties. The cell adhesion-inhibiting material, which is decomposed or denatured by action of the photocatalyst on the basis of the irradiation with the energy, may be one described in the item “1. Cell Adhesion-Inhibiting Material” in “A. Coating Liquid for Patterning Substrate”.

In the embodiment, the cell adhesion-inhibiting layer comprises therein the binder; therefore, for example, even if the cell adhesion-inhibiting material is completely decomposed by action of a photocatalyst on the basis of irradiation with energy, the binder remains and the cell adhesion-inhibiting layer remains. The binder is not limited to any special kind if the binder has no cell adhesion-inhibiting properties, and may be a binder which is ordinarily used in a cell culture layer or the like of a cell culture substrate.

In the embodiment also, the cell adhesion-inhibiting layer preferably comprises a cell adhesive material. This makes it possible that when the cell adhesion-inhibiting layer is irradiated with energy, the cell adhesive properties of the region irradiated with the energy are made better so that cells are caused to adhere, in a more highly precise pattern, only onto the region irradiated with the energy.

In the embodiment, the cell adhesion-inhibiting layer can be formed, for example, by applying a coating liquid comprising the cell adhesion-inhibiting material and the binder. In the case of incorporating a cell adhesive material into the layer as described above, the layer can be formed by using a coating liquid for patterning substrate comprising the cell adhesive material besides the cell adhesion-inhibiting material, as described in the item “A. Coating Liquid for Patterning Substrate”.

The method for forming this cell adhesion-inhibiting layer, the film thickness of the cell adhesion-inhibiting layer, and others are equivalent to those in the first embodiment. Thus, detailed description thereof is omitted herein. The cell adhesive material and the binder used in the cell adhesion-inhibiting layer, the cell adhesion-inhibiting material, and others are equivalent to those in the item “A. Coating Liquid for Patterning Substrate”. Thus, description thereof is omitted herein.

About the cell adhesion-inhibiting layer in the embodiment also, it is preferred that the layer does not comprise any photocatalyst. This makes it possible that, for example, when cells are caused to adhere onto the cell adhesion-inhibiting layer, the cells are prevented from being affected by any photocatalyst with the passage of time to make the quality of the embodiment high.

(Patterning Substrate)

The following will describe the patterning substrate of the present embodiment. The patterning substrate is not limited to any special kind if the patterning substrate is a member wherein the cell adhesion-inhibiting layer is formed on the base material. If necessary, other layers, such as a light-shielding layer, may be laminated thereon.

C. Cell Culture Patterning Substrate

The following will describe the cell culture patterning substrate of the present invention. This cell culture patterning substrate is a substrate wherein the cell adhesion-inhibiting layer of the above-mentioned patterning substrate comprises a cell adhesion portion where the cell adhesion-inhibiting material is decomposed or denatured in a pattern form, and a cell adhesion-inhibiting portion which is a region other than the cell adhesion portion.

As shown in, for example, FIG. 3, the cell culture patterning substrate of the present invention has a base material 1, and a cell adhesion-inhibiting layer 2 formed on the base material 1, wherein the cell adhesion-inhibiting layer 2 has cell adhesion portions 4 having cell adhesive properties, where the cell adhesion-inhibiting material comprised in the layer 2 is decomposed or denatured, and cell adhesion-inhibiting portions 2′ having cell adhesion-inhibiting properties, which are regions other than the cell adhesion portions 4.

Since the cell culture patterning substrate of the invention has the cell adhesion portions and the cell adhesion-inhibiting portions, cells can easily be caused to adhere only onto the cell adhesion portions. For example, even if cells are applied onto the entire surface of the cell adhesion-inhibiting layer, the cells can be caused to adhere highly precisely only onto the cell adhesion portions.

As shown in, for example, FIG. 4, in the invention, light-shielding portions 3 may be formed, on a base material 1, in the same pattern as the cell adhesion-inhibiting portions 2′ of the cell adhesion-inhibiting layer 2 have. Such formation of the light-shielding portions makes the following possible: for example, in the case of forming the patterning substrate described in the item “B. Patterning Substrate”, arranging the cell adhesion-inhibiting layer and a photocatalyst-containing layer comprising a photocatalyst to face to each other, and then irradiating energy onto the resultant from the base material side thereof, the cell adhesion-inhibiting material only on the region where the light-shielding portions are not formed is decomposed or denatured so that the cell adhesion portions are easily formed.

The cell adhesion-inhibiting portions are regions which are low in adhesive properties onto target cells and comprise a cell adhesion-inhibiting material which has cell adhesion-inhibiting properties of inhibiting adhesion onto cells and is decomposed to remove or denatured by action of a photocatalyst on the basis of irradiation with energy.

On the other hand, the cell adhesion portions are regions having cell adhesive properties where the cell adhesion-inhibiting material is decomposed or denatured. The wording “the cell adhesion-inhibiting material is decomposed or denatured” means that the cell adhesion-inhibiting material is not contained or the cell adhesion-inhibiting material is contained in a smaller amount than the amount of the cell adhesion-inhibiting material contained in the cell adhesion-inhibiting portions. When the cell adhesion-inhibiting material is, for example, a material which can be decomposed by action of a photocatalyst on the basis of irradiation with energy, the wording means that the cell adhesion portions do not comprise therein the cell adhesion-inhibiting material or comprise a decomposition product of the cell adhesion-inhibiting material, or the like. When the cell adhesion-inhibiting material is a material which can be decomposed, it is preferred that a binder or the like is normally comprised in the cell adhesion-inhibiting layer and the layer is made into cell adhesion portions having cell adhesive properties by making the binder exposed, or the like. On the other hand, when the cell adhesion-inhibiting material is a material which can be denatured by action of a photocatalyst on the basis of irradiation with energy, a denatured product thereof or the like is comprised in the cell adhesion portions. In this case, the cell adhesion portions can have cell adhesive properties by action of the denatured product of the cell adhesion-inhibiting material. Consequently, the cell adhesion-inhibiting layer may comprise therein a binder or the like other than the cell adhesion-inhibiting material, or may not comprise therein any binder or the like.

In any case in the invention, it is particularly preferred that the cell culture patterning substrate comprises a cell adhesive material good in cell adhesive properties, as described in the item “A. Coating Liquid for Patterning Substrate”. This makes it possible to make the cell adhesive properties of the cell adhesion portions better.

The base material and the cell adhesion-inhibiting layer used in the cell culture patterning substrate of the invention are equivalent to those described in the item “B. Patterning Substrate”. Thus, description thereof is omitted herein. The following will describe a method for forming the cell adhesion-inhibiting layer having the cell adhesion portions and the cell adhesion-inhibiting portions.

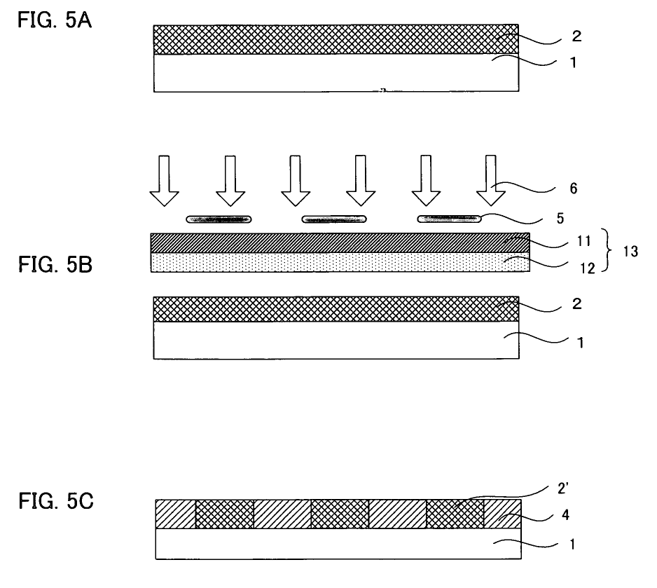

As shown in, for example, FIGS. 5A to 5C, the coating liquid for patterning substrate described in the item “A. Coating Liquid for Patterning Substrate” or the like is first used to form a cell adhesion-inhibiting layer 2 comprising at least a cell adhesion-inhibiting material on a base material 1 (FIG. 5A). Next, prepared is a photocatalyst-containing layer side substrate 13 having a base body 11 and a photocatalyst-containing layer 12 which is formed on the base body 11 and comprises at least a photocatalyst. This photocatalyst-containing layer 12 and the cell adhesion-inhibiting layer 2 are arranged to be facing each other (FIG. 5B). Subsequently, for example, a photomask 5 or the like is used to irradiate energy 6 into a pattern form for forming cell adhesion portions (FIG. 5B). This is permitted to form cell adhesion portions 4 having cell adhesive properties, where the cell adhesion-inhibiting material comprised in the cell adhesion-inhibiting layer 2 is decomposed or denatured by action of the photocatalyst contained in the photocatalyst-containing layer 12, and cell adhesion-inhibiting portions 2′ low in cell adhesive properties, where the cell adhesion-inhibiting material is not decomposed since the irradiation of the energy is not received (FIG. 5C). The method for forming the cell adhesion-inhibiting layer or the like is equivalent to the method described in the item “B. Patterning Substrate”. Thus, description thereof is omitted herein. Hereinafter, the photocatalyst-containing layer side substrate, which has the photocatalyst-containing layer, the energy, and others that are used to form such cell adhesion portions will be described.

(Photocatalyst-Containing Layer Side Substrate)

First, the photocatalyst-containing layer side substrate, which has a photocatalyst-containing layer comprising a photocatalyst, is described. The photocatalyst-containing layer side substrate used in the invention is normally a substrate having a photocatalyst-containing layer comprising a photocatalyst, and is usually a base body and substrate wherein a photocatalyst-containing layer is formed on the base body. This photocatalyst-containing layer side substrate may have, for example, photocatalyst-containing layer side light-shielding portions formed in a pattern form, a primer layer, or the like. The following will describe each of the constituents of the photocatalyst-containing layer side substrate used in the present process.

a. Photocatalyst-Containg Layer

First, the photocatalyst-containing layer used in the photocatalyst-containing layer side substrate is described. The photocatalyst-containing layer is not limited to any special structure if the layer has a structure wherein the photocatalyst contained in this layer causes the decomposition or denaturation of the cell adhesion inhibiting material in the adjacent or near cell adhesion-inhibiting layer. The photocatalyst-containing layer may be a film composed of the photocatalyst and a binder, or may be a film made only of the photocatalyst. The property of the surface thereof may be lyophilic or repellent to liquid.

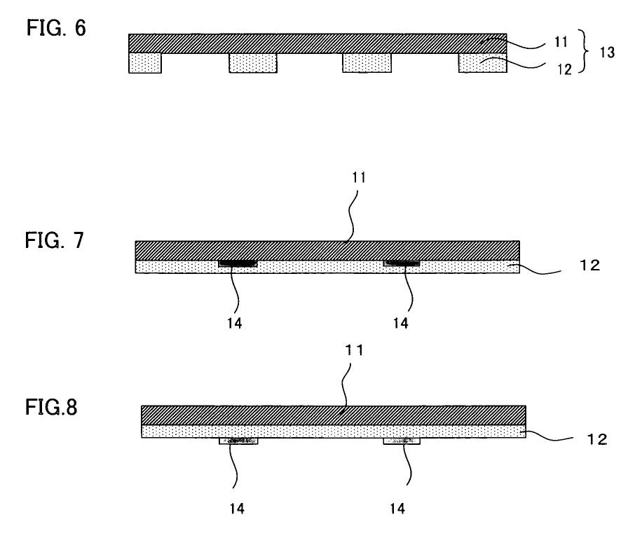

As shown in, for example, FIG. 5B, the photocatalyst-containing layer used in the invention may be formed on the entire surface of a base body 11. Alternatively, as shown in, for example, FIG. 6, a photocatalyst-containing layer 12 may be formed in a pattern form on a base body 11.

The formation of the photocatalyst-containing layer in a pattern in this way makes it unnecessary to perform patterning irradiation using a photomask or the like when energy is irradiated onto the patterning substrate to form cell adhesion portions. Thus, the irradiation thereof onto the entire surface is permitted to form a pattern wherein the cell adhesion-inhibiting material contained in the cell adhesion-inhibiting layer is decomposed or denatured.

The method for patterning the photocatalyst-containing layer is not particularly limited. For example, a method such as a photolithography may be used.

The cell adhesion-inhibiting material only on the cell adhesion-inhibiting layer facing the photocatalyst-containing layer is actually decomposed or denatured; therefore, the direction in which energy is irradiated may be any direction if the energy is irradiated onto the area where the photocatalyst-containing layer and the cell adhesion-inhibiting layer face each other. Furthermore, there is generated an advantage that the irradiated energy is not limited to energy composed of parallel components, such as parallel light rays.

As the photocatalyst that can be used in the present invention, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present invention, in particular, titanium dioxide, owing to its large band gap, chemical stability, non-toxicity, and easy availability, can be preferably used. There are two types of titanium dioxide, anatase type and rutile type, and both can be used in the invention; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-K01, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. An average particle diameter of the photocatalyst is preferably 50 nm or less, and one having an average particle diameter of 20 nm or less can be particularly preferably used.

The photocatalyst-containing layer in the invention may be made only of the photocatalyst or made of a mixture of the photocatalyst and a binder, as described above.

In the case of the photocatalyst-containing layer made only of the photocatalyst, the efficiency of the decomposition or denaturation of the cell adhesion-inhibiting material in the cell adhesion-inhibiting layer is improved so that costs decrease because of a reduction in the treating time, and so on. On the other hand, the case of the photocatalyst-containing layer composed of the photocatalyst and the binder has an advantage that this layer is easily formed.

The method for forming the photocatalyst-containing layer made only of the photocatalyst may be, for example, a method using a vacuum film-forming process such as sputtering, CVD, or vacuum vapor deposition. The formation of the photocatalyst-containing layer by vacuum vapor deposition makes it possible to render the layer a homogeneous photocatalyst-containing layer containing only the photocatalyst. According to this, the following becomes possible: the cell adhesion-inhibiting material in the cell adhesion-inhibiting layer is homogeneously decomposed or denatured; and the cell adhesion-inhibiting material is more efficiently decomposed or denatured in this case than in the case of using the binder also since only the photocatalyst is used in the former case.

Another example of the method for forming the photocatalyst-containing layer made only of a photocatalyst, is the following method: in the case that the photocatalyst is, for example, titanium dioxide, amorphous titania is formed on the base body and next fired so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is fired at 400 to 500° C. so as to be denatured to anatase type titania, and fired at 600 to 700° C. so as to be denatured to rutile type titania.

In the case of using a binder together with the above-mentioned photocatalyst, the binder is preferably a binder having a high bonding energy, wherein its main skeleton is not decomposed by photoexcitation of the photocatalyst. Examples of such a binder include the organopolysiloxanes or the like.

In the case of using such an organopolysiloxane as the binder, the photocatalyst-containing layer can be formed by dispersing a photocatalyst, the organopolysiloxane as the binder, and optional additives if needed into a solvent to prepare a coating liquid, and applying this coating liquid onto the base body. The used solvent is preferably an alcoholic based organic solvent such as ethanol or isopropanol. The application can be performed by a known coating method such as spin coating, spray coating, dip coating, roll coating, or bead coating. When the coating liquid contains an ultraviolet curable component as the binder, the photocatalyst-containing layer can be formed by curing the coating liquid through the irradiation of ultraviolet rays onto the liquid.

As the binder, an amorphous silica precursor, a surfactant or the like may be used. Such a material may be the same as described in JP-A No. 2000-249821.

b. Base Body

The following will describe the base body used in the photocatalyst-containing layer side substrate. As shown in FIG. 5B, in the invention, the photocatalyst-containing layer side substrate has at least a base body 11, and a photocatalyst-containing layer 12 formed on this base body 11. In this case, the material which constitutes the used base body is appropriately selected in accordance with the direction of energy irradiation which will be detailed later, the matter as to whether or not transparency is necessary for the pattern-formed body to be yielded, or other factors.

The base body used in the invention may be a member having flexibility, such as a resin film, or may be a member having no flexibility, such as a glass plate. This is appropriately selected in accordance with the method for the energy irradiation.

An anchor layer may be formed on the base body in order to improve the adhesive property between the base body surface and the photocatalyst-containing layer. The anchor layer may be made of, for example, a silane-based or titanium-based coupling agent.

c. Photocatalyst-Containing Layer Side Light-Shielding Portion

The photocatalyst-containing layer side substrate used in the invention may be a photocatalyst-containing layer side substrate on which photocatalyst-containing layer side light-shielding portions may be formed into a pattern form. When the photocatalyst-containing layer side substrate having photocatalyst-containing layer side light-shielding portions is used in this way, it is unnecessary to use, at the time of irradiating energy, any photomask or to irradiate a laser ray for drawing irradiation. It is therefore unnecessary to position a photomask precisely onto the photocatalyst-containing layer side substrate. Consequently, it is unnecessary to use any complicated step or any expensive device for drawing irradiation, thereby producing an advantage for costs.

Such a photocatalyst-containing layer side substrate having photocatalyst-containing layer side light-shielding portions can be classified into the following two embodiments, depending on the position where the photocatalyst-containing layer light-shielding portions are formed.

One of them is an embodiment, for example, as shown in FIG. 7, wherein photocatalyst-containing layer side light-shielding portions 14 are formed on a base body 11 and a photocatalyst-containing layer 12 is formed on the photocatalyst-containing layer side light-shielding portions 14. The other example is an embodiment as shown in, for example, FIG. 8, wherein a photocatalyst-containing layer 12 is formed on a base body 11 and photocatalyst-containing layer side light-shielding portions 14 are formed on the layer 12.

In any one of the embodiments, the effect of energy-scattering in the base body or the like can be made smaller than in the case of using a photomask since the photocatalyst-containing layer side light-shielding portions are arranged near the region where the photocatalyst-containing layer and the cell adhesion-inhibiting layer are arranged. Accordingly, irradiation of energy in a pattern form can be extremely precisely attained.

In the invention, an embodiment wherein photocatalyst-containing layer side light-shielding portions 14 are formed on a photocatalyst-containing layer 12 as shown in FIG. 8 has an advantage that at the time of arranging the photocatalyst-containing layer and the cell adhesion-inhibiting layer in position, the film thickness of the light-shielding portions 14 can be made consistent with the width of the interval between the two layers, thereby using the light-shielding portions 14 as a spacer for making the interval constant.

In other words, when the photocatalyst-containing layer and the cell adhesion-inhibiting layer are arranged to face to each other so as to have a given interval therebetween, the dimension of the interval can be made accurate by arranging the photocatalyst-containing layer side light-shielding portions and the cell adhesion-inhibiting layer in the state that they are caused to adhere closely to each other. By irradiating the resultant with energy in this state, the cell adhesion-inhibiting material is not decomposed or denatured in the regions of the cell adhesion-inhibiting layer which contact the light-shielding portions, so that cell adhesion portions can be formed with a sufficient precision.

The method for forming such photocatalyst-containing layer side light-shielding portions is not particularly limited, and may be appropriately selected in accordance with the property of the face where the light-shielding portions are to be formed, power for shielding required energy, and others. The light-shielding portions may be the same light-shielding portions as described in the item “B. Patterning Substrate” which are formed on a base material. Thus, detailed description thereof is omitted herein.

The above has described two cases, wherein the photocatalyst-containing layer side light-shielding portions are formed between the base body and the photocatalyst-containing layer and are formed on the surface of the photocatalyst-containing layer. Besides, the photocatalyst-containing layer side light-shielding portions may be formed on the base body surface on which no photocatalyst-containing layer is formed. In this embodiment, for example, a photomask can be made close to this surface to such a degree that the photomask can be put on and taken off. Thus, the embodiment can be preferably used for the case that the pattern of the cell adhesion-inhibiting portions is varied for every small lot.

d. Primer Layer

The following will describe a primer layer used in the photocatalyst-containing layer side substrate of the invention. When photocatalyst-containing layer side light-shielding portions are formed into a pattern form on a base body and a photocatalyst-containing layer is formed thereon in the invention so as to prepare a photocatalyst-containing layer side substrate described above, a primer layer may be formed between the photocatalyst-containing layer side light-shielding portions and the photocatalyst-containing layer.

The effect and function of this primer layer are not necessarily clear, but would be as follows: the primer layer is formed between the photocatalyst-containing layer side light-shielding portions and the photocatalyst-containing layer, whereby the primer layer exhibits a function of preventing the diffusion of impurities from openings which are present in and between the light-shielding portions, the impurities being factors for blocking the decomposition or denaturation of the cell adhesion-inhibiting material by action of the photocatalyst, in particular, residues generated when the photocatalyst-containing layer side light-shielding portions are patterned, or metal, metal ion impurities, or the like. Accordingly, the formation of the primer layer makes it possible that the processing of the decomposition or denaturation of the cell adhesion-inhibiting material advances with high sensitivity so as to yield cell adhesion portions which are highly precisely formed.

The primer layer in the invention is a layer for preventing the effect of the photocatalyst from being affected by the impurities present inside not only the photocatalyst-containing layer side light-shielding portions but also the openings made between the light-shielding portions. It is therefore preferred to form the primer layer over the entire surface of the light-shielding portions plus the openings.

The primer layer in the invention is not limited to any special structure if the primer layer is formed not to bring the photocatalyst-containing layer side light-shielding portions and the photocatalyst-containing layer of the photocatalyst-containing layer side substrate into contact with each other.

A material that forms the primer layer, though not particularly restricted, is preferably an inorganic material that is not likely to be decomposed by action of the photocatalyst. Specifically, amorphous silica can be cited. When such amorphous silica is used, a precursor of the amorphous silica is preferably a silicon compound that is represented by a general formula, SiX₄, X being halogen, methoxy group, ethoxy group, acetyl group or the like, silanol that is a hydrolysate thereof, or polysiloxane having an average molecular weight of 3000 or less.

A film thickness of the primer layer is preferably in the range of 0.001 to 1 μm and particularly preferably in the range of 0.001 to 0.1 μm.

(Irradiation of Energy)

The following will describe the irradiation of energy when the cell adhesion portions are formed. The irradiation of energy can be performed by arranging the above-mentioned cell adhesion-inhibiting layer and the photocatalyst-containing layer of the above-mentioned photocatalyst-containing layer side substrate to have a given interval therebetween and giving the energy to the resultant from a given side.

The above-mentioned wording “arranging” means that the above-mentioned two layers; the photocatalyst-containing layer and the cell adhesion-inhibiting layer are arranged in the state that the action of the photocatalyst can substantially work to the surface of the cell adhesion-inhibiting layer, and include not only the state that the two layers physically contact each other, but also the state that the two layers are arranged at a given interval. The dimension of the interval is preferably 200 μm or less.

The dimension of the above-mentioned interval in the invention is more preferably from 0.2 to 10 μm, even more preferably from 1 to 5 μm since the precision of the pattern to be obtained becomes very good and further the sensitivity of the photocatalyst becomes high so as to make good the efficiency of the decomposition or denaturation of the cell adhesion-inhibiting material in the cell adhesion-inhibiting layer. This range of the interval dimension is particularly effective for the cell adhesion-inhibiting layer small in area, which makes it possible to control the interval dimension with a high precision.

Meanwhile, in the case of irradiating energy onto the cell adhesion-inhibiting layer large in area, for example, 300 mm or more×300 mm or more in size, it is very difficult to make a fine interval as described above between the photocatalyst-containing layer side substrate and the cell adhesion-inhibiting layer without contacting each other. Accordingly, when the cell adhesion-inhibiting layer has a relatively large area, the interval dimension is preferably from 10 to 100 μm, more preferably from 50 to 75 μm. The limitation of the interval dimension into this range gives an advantageous effect that the cell adhesion-inhibiting material is not unevenly decomposed or denatured without causing problems based on a fall in patterning precision, such as a problem that a blurred pattern is obtained, or other problems, such as a problem that the sensitivity of the photocatalyst deteriorates so that the efficiency of decomposing or denaturing the cell adhesion-inhibiting material also deteriorates.

When energy is irradiated onto the cell adhesion-inhibiting layer having a relatively large area as described above from an energy irradiating device, a unit for positioning the photocatalyst-containing layer side substrate and the cell adhesion-inhibiting layer inside the device is permitted to set the dimension of the interval therebetween preferably into the range of 10 to 200 μm, more preferably into the range of 25 to 75 μm. The setting of the interval dimension value into this range makes it possible to arrange the photocatalyst-containing layer side substrate and the cell adhesion-inhibiting layer without causing a large drop in patterning precision or in sensitivity of the photocatalyst, or bringing the substrate and the layer into contact with each other.

When the photocatalyst-containing layer and the surface of the cell adhesion-inhibiting layer are arranged at a given interval as described above, active oxygen species generated from oxygen and water by action of the photocatalyst can easily be released. In other words, if the interval between the photocatalyst-containing layer and the cell adhesion-inhibiting layer is made narrower than the above-mentioned range, the active oxygen species are not easily released, so as to make the rate for decomposing or denaturing the cell adhesion-inhibiting material unfavorably small. If the two layers are arranged at an interval larger than the above-mentioned range, the generated active oxygen species do not reach the cell adhesion-inhibiting layer easily. In this case also, the rate for decomposing or denaturing the cell adhesion-inhibiting material unfavorably becomes small.

The method for arranging the photocatalyst-containing layer and the cell adhesion-inhibiting layer so as to form such a very small interval homogeneously therebetween is, for example, a method of using spacers. The use of the spacers in this way makes it possible to make a homogeneous interval. In regions which the spacers contact, the action of the photocatalyst does not work onto the surface of the cell adhesion-inhibiting layer; therefore, when the spacers are rendered spacers having a pattern similar to that of the cell adhesion-inhibiting portions, the cell adhesion-inhibiting material only portions where no spacers are formed can be decomposed or denatured so that highly precise cell adhesion portions can be formed. The use of the spacers also makes it possible that the active oxygen species generated by action of the photocatalyst reach the surface of the cell adhesion-inhibiting layer, without diffusing, at a high concentration. Accordingly, highly precise cell adhesion portions can be effectively formed.

In the invention, it is sufficient that such an arrangement state of the photocatalyst-containing layer side substrate is maintained only during the irradiation of energy.

The energy irradiation (exposure) mentioned in the present invention is a concept that includes all energy line irradiation that can decompose or denature the cell adhesion-inhibiting material by action of a photocatalyst on the basis of irradiation with energy, and is not restricted to light irradiation.

Normally, a wavelength of light used in such energy irradiation is set in the range of 400 nm or less, and preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

The method for the exposure may be a method of using a laser, such as an excimer laser or YAG laser, to draw and irradiate energy in a pattern form, besides a method of using a light source as described above to draw and irradiate energy in a pattern form through a photomask. When the base material has light-shielding portions in the same pattern form as the cell adhesion-inhibiting portions have, as described above, the exposure can be performed by irradiating the entire surface from the base material side thereof. When the photocatalyst-containing layer has thereon light-shielding portions in the same pattern form as the cell adhesion-inhibiting portions have, the exposure can be performed by irradiating the entire surface from any direction. These cases have an advantage that a photomask, and positioning and other steps are unnecessary.

When the base material has one or more concave portions and the cell adhesion-inhibiting layer is formed in the concave portion(s) as described above, energy may be irradiated onto the entire surface thereof by the above-mentioned method. In the case of the plural concave portions, for example, the exposure may be performed into the form of patterns different from each other for the individual concave portions. Examples of the method for performing the exposure into the form of patterns different from each other for the individual concave portions as described above include a method of arranging different masks for the individual concave portions to irradiate energy by use of the above-mentioned photocatalyst-containing layer side substrate; and a method of arranging the photocatalyst-containing layer side substrate and a chromium mask, a stencil mask or the like at the tip of an optical fiber to irradiate energy.

In order not to irradiate any energy onto side walls of the concave portions, the method for the exposure may be, for example, a method of using a cylindrical mask to irradiate energy only onto the bottom faces of the concave portions.

The irradiation quantity of the energy at the time of the energy-irradiation is set to a value necessary for decomposing or denaturing the cell adhesion-inhibiting material by action of the photocatalyst.

In this case, it is preferred to irradiate the photocatalyst-containing layer with the energy while heating the layer since the sensitivity can be raised so as to decompose or denature the cell adhesion-inhibiting material effectively. Specifically, the layer is preferably heated at a temperature of 30 to 80° C.

When the base material is transparent, the irradiation of energy through a photomask may be performed from either of the side of the base material and the side of the photocatalyst-containing layer side substrate. On the other hand, when the base material is opaque, energy needs to be irradiated from the side of the photocatalyst-containing layer side substrate.

(Cell Culture Patterning Substrate)

The following will describe the cell culture patterning substrate of the present invention. The cell culture patterning substrate is not limited to any special kind if the substrate is a member wherein a cell adhesion-inhibiting layer comprising the cell adhesion portion and the cell adhesion-inhibiting portion is formed on the base material, each of these constituents being defined above. If necessary, one or more different layers may be appropriately formed in the patterning substrate.

In the invention, the cell culture patterning substrate may be a member obtained by forming the above-mentioned cell adhesion portion, cutting one portion from the resultant cell culture patterning substrate, and attaching this portion onto the bottom or other part of a base material in a concave form.

D. Cell Culture Substrate

Next, a cell culture substrate in the invention will be explained. A cell culture substrate in the invention is one in which cells are adhered onto the cell adhesion portion in the above-mentioned cell culture patterning substrate.

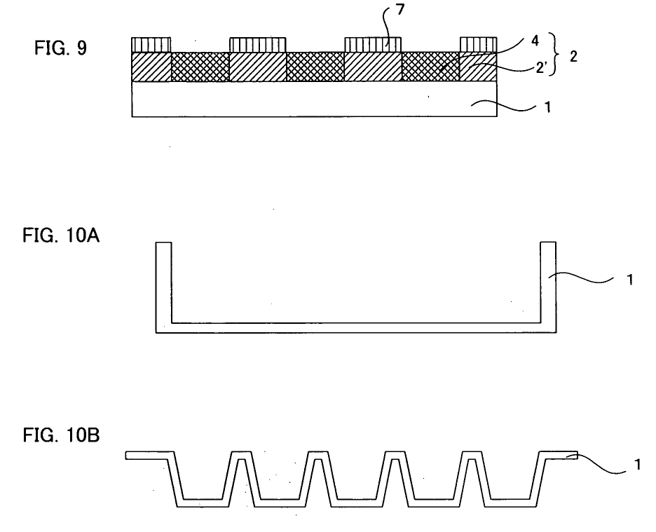

In the cell culture substrate in the invention, as shown in, for example, FIG. 9, cells 7 are adhered only onto a cell adhesion portion 4 of the cell adhesion-inhibiting layer 2, and, on a cell adhesion inhibiting portion 2′, cells 7 are not adhered.

According to the invention, the cell adhesion portions having cell adhesive properties and the cell adhesion-inhibiting portions having no cell adhesive properties are formed on the cell culture patterning substrate; for example, therefore, even if cells are applied onto the entire surface of the cell culture patterning substrate, the cells are caused to adhere only onto the cell adhesion portions and the cells on the cell adhesion-inhibiting portions can easily be removed. This makes it possible to form a cell pattern easily without using any complicated step or any treating solution or the like that produces a bad effect on the cells. Since the cell culture patterning substrate does not need to comprise therein any photocatalyst, the cells therein are not affected by any photocatalyst with the passage of time. Thus, the present cell culture substrate is a high-quality cell culture substrate.

Hereinafter, cells that are used in a cell culture substrate in the invention will be explained. Since an explanation of a cell culture patterning substrate is the same as that in the “C. Cell Culture Patterning Substrate”, here it is omitted.

(Cells)

As cells used in a cell culture substrate in the invention, as far as cells can adhere onto a cell adhesion portion of the cell culture patterning substrate but do not adhere to a cell adhesion-inhibiting portion, there is no particular restriction.

As cells used in the present invention, except for, for instance, non-adhesive cells such as nervous tissue, liver, kidney, pancreas, blood vessel, brain, cartilage and blood corpuscle, all tissues present in an organism and cells derived therefrom can be used. Furthermore, since even for generally non-adhesive cells, recently, in order to adhere and fix, a technology of modifying a cell membrane is devised; accordingly, as needs arise, the non-adhesive cells, when this technology is applied, can be used in the present invention.

The respective tissues such as mentioned above are formed of cells having various functions; accordingly, it is necessary to select desired cells to use. For instance, in the case of the liver, it is formed of, other than hepatocytes, epithelial cells, endothelial cells, Kupffer's cells, fibroblasts, and fat-storing cells and the like. In this case, since the cell adhesion-inhibiting properties with a cell adhesion-inhibiting material is different depending on the kinds of the cells, in accordance with a cell strain, a cell adhesion-inhibiting material used in the cell adhesion-inhibiting portion and a composition ratio thereof have to be selected.

A method of adhering cells to the cell adhesion portion, as far as it can adhere cells only on the cell adhesion portion of the cell culture patterning substrate that has the cell adhesion portion and the cell adhesion-inhibiting portion, is not particularly restricted. For instance, cells may be adhered by use of an ink jet printer, a manipulator or the like; however, a method in which after a cell suspension is disseminated to adhere cells on the cell adhesion portion, unnecessary cells on a cell adhesion-inhibiting portion are washed with a phosphate buffer to remove the cells is generally used. As such a method, a method described in, for instance, a reference literature, Kevin E. Healy et al., “Spatial distribution of mammalian cells dicated by material surface chemistry”, Biotech. Bioeng. (1994), p. 792 can be used.

E. Method for Producing a Cell Culture Patterning Substrate

The following will describe a method of the invention for producing a cell culture patterning substrate. The method for producing a cell culture patterning substrate comprises: a cell adhesion-inhibiting layer forming process of forming, on a base material, a cell adhesion-inhibiting layer comprising a cell adhesion-inhibiting material that has cell adhesion-inhibiting properties of inhibiting adhesion to cells and is decomposed or denatured by action of a photocatalyst on the basis of irradiation with energy; and

an energy irradiating process of arranging the cell adhesion-inhibiting layer and a photocatalyst-containing layer side substrate having a photocatalyst-containing layer comprising the photocatalyst and a base body to dispose the cell adhesion-inhibiting layer and the photocatalyst-containing layer to face to each other, and then irradiating energy onto the resultant from a given direction to form a pattern composed of a cell adhesion portion wherein the cell adhesion-inhibiting material comprised in the cell adhesion-inhibiting layer is decomposed or denatured, and a cell adhesion-inhibiting portion which is other than the cell adhesion portion.

As shown in, for example, FIGS. 5A to 5C, the method of the invention for producing a cell culture patterning substrate comprises a cell adhesion-inhibiting layer forming process of forming, on a base material 1, a cell adhesion-inhibiting layer 2 comprising at least a cell adhesion-inhibiting material (FIG. 5A), and an energy irradiating process of preparing a photocatalyst-containing layer side substrate 13 having a base body 11 and a photocatalyst-containing layer 12 comprising a photocatalyst, arranging the cell adhesion-inhibiting layer 2 and the photocatalyst-containing layer 12 to be facing each other, and irradiating energy 6 onto the resultant through a photomask 5 or the like(FIG. 5B) to form cell adhesion portions 4 where the cell adhesion-inhibiting material contained in the cell adhesion-inhibiting layer 2 inside the regions irradiated with the energy is decomposed or denatured, and cell adhesion-inhibiting portions 2′ low in cell adhesive properties, which are not irradiated with the energy (FIG. 5C).

According to the invention, by using the photocatalyst-containing layer side substrate to perform the irradiation of the energy, the cell adhesion-inhibiting material contained in the cell adhesion-inhibiting layer can easily be decomposed or denatured to form cell adhesion portions and cell adhesion-inhibiting portions in a highly precise pattern form. Since the cell adhesion-inhibiting layer does not need to comprise therein any photocatalyst, it is possible to produce a high-quality cell culture patterning substrate which is not affected by any photocatalyst with the passage of time.

The following will describe each of the processes in the method of the invention.

1. Cell Adhesion-Inhibiting Layer Forming Process

First, the cell adhesion-inhibiting layer forming process in the invention is described. The cell adhesion-inhibiting layer forming process is a process of forming, on a base material, a cell adhesion-inhibiting layer comprising a cell adhesion-inhibiting material that has cell adhesion-inhibiting properties of inhibiting adhesion to cells and is decomposed or denatured by action of a photocatalyst on the basis of irradiation with energy. In the invention, the process can be performed by applying a coating liquid comprising the above-mentioned cell adhesion-inhibiting material, or the like onto a base material. The method for the application, constituent materials of the coating liquid and others are not particularly limited. It is preferred that the coating liquid comprises a cell adhesive material having good cell adhesive properties at least after the material is irradiated with energy. This makes it possible that in the energy irradiating process, which will be detailed later, the cell adhesive properties of a cell adhesion portion, which is a region irradiated with energy, are made high, so as to produce a cell culture patterning substrate onto which cells can be caused to adhere in a highly precise pattern form.

The cell adhesion-inhibiting material, the cell adhesive material and the base material used in the present process, and the method for forming a cell adhesion-inhibiting layer are equivalent to those described in the item “B. Patterning Substrate”. Thus, description thereof is omitted herein.

2. Energy Irradiating Process

Secondly, the energy irradiating process in the invention is described. The energy irradiating process is a process of arranging the cell adhesion-inhibiting layer and a photocatalyst-containing layer side substrate having a photocatalyst-containing layer comprising a photocatalyst and a base body to dispose the cell adhesion-inhibiting layer and the photocatalyst-containing layer to face to each other, and then irradiating energy onto the resultant from a given direction to form a pattern composed of a cell adhesion-inhibiting portion wherein the cell adhesion-inhibiting material comprised in the cell adhesion-inhibiting layer is decomposed or denatured, and a cell adhesion portion which is other than the cell adhesion-inhibiting portion.

The method and so on for the present process are not particularly limited if they make it possible to dispose the cell adhesion-inhibiting layer formed in the cell adhesion-inhibiting layer forming process and the photocatalyst-containing layer of the photocatalyst-containing layer side substrate to face to each other, and irradiate energy in a pattern form onto the resultant so as to decompose or denature the cell adhesion material in the cell adhesion-inhibiting layer, thereby forming a cell adhesion portion which has cell adhesive properties.

The photocatalyst-containing layer side substrate and the energy irradiating method used in the present process are equivalent to those described in the item “C. Cell Culture Patterning Substrate”. Thus, detailed description thereof is omitted herein.

F. Method for Producing a Cell Culture Substrate

The following will describe the method of the invention for producing a cell culture substrate. This method comprises a cell adhesion process of causing cells to adhere onto the cell adhesion portion of the cell culture patterning substrate produced by the above-mentioned cell culture patterning substrate producing method.

According to the present invention, in the cell culture patterning substrate, the cell adhesion portion having cell adhesive properties and the cell adhesion-inhibiting portion having no cell adhesive properties are formed in the cell adhesion-inhibiting layer; therefore, for example, when cells are applied onto this cell adhesion-inhibiting layer, a cell culture substrate wherein the cells adhere only onto the cell adhesion portion can easily be produced. The cell culture patterning substrate does not need to comprise therein any photocatalyst; therefore, the cell culture substrate can be rendered a high-quality cell culture substrate wherein the cells are not affected by any photocatalyst with the passage of time.

The manner for the cell adhesion process in the present embodiment is not limited to any special manner if the manner is a manner capable of causing cells to adhere onto the cell adhesion portion. The manner may be a manner as described in the item “D. Cell Culture Substrate”. Thus, detailed description thereof is omitted herein.

The present invention is not limited to the above-mentioned embodiments. The embodiments are mere examples. All modifications which have substantially the same structure as the technical conception described in the claims of the invention and produce effects and advantages similar to those of the technical conception are included in the technical scope of the invention.

### EXAMPLES

Hereinafter, examples are shown and thereby the present invention will be more specifically described.

### Example 1

(Formation of a Cell Adhesion-Inhibiting Layer)

Three grams of isopropyl alcohol, 0.4 g of an organosilane, TSL8114 (manufactured by GE Toshiba Silicones), and 0.1 g of a fluoroalkylsilane, XC95-A9715 (manufactured by GE Toshiba Silicones) were mixed, and the mixture was heated at 100° C. for 20 minutes while stirred. This solution was applied onto a glass substrate (thickness: 0.7 mm) subjected to alkali treatment in advance by spin coating, and the substrate was dried at 150° C. for 10 minutes to advance hydrolysis and polycondensation reaction, thereby forming, on the substrate, a cell adhesion-inhibiting layer (thickness: about 40 nm) made of an organopolysiloxane layer having fluoroalkyl groups. In this way, a patterning substrate was formed.

(Patterning of the Patterning Substrate)

A photomask on which a photocatalyst layer was formed was put onto the patterning substrate to dispose the photocatalyst layer and the cell adhesion inhibiting layer of the patterning substrate to face to each other, and then ultraviolet rays were irradiated from a mercury lamp through the photomask onto this patterning substrate at 6 J/cm² (measured wavelength: 254 nm) to yield a cell culture patterning substrate having a cell adhesive surface patterned so as to have unexposed portions having cell adhesion-inhibiting properties and exposed portions having cell adhesive properties.

(Cell Adhesion Process)

About the process of experiments for culturing cells originating from various kinds of tissues, details thereof are described in, for example “Soshikibaiyo no Gijyutsu, Dai San Han, Kiso”, edited by The Japanese Tissue Culture Association and published by Asakura Shoten, and other documents.

In the present application, rat hepatocytes were used to evaluate the cell culture patterning substrate.

A liver extracted from a rat was transferred into a Petri dish, and the liver was cut into pieces 5 mm in size with a scalpel. Thereto was added 20 ml of a DMEM culture medium, and the pieces were lightly suspended with a pipette. Thereafter, the suspension was filtrated with a cell filter. The resultant cell-coarsely-dispersed suspension was subjected to centrifugation at 500 to 600 rpm for 90 seconds, and the supernatant was sucked to be removed. A new DMEM culture medium was added to the remaining cells, and the resultant was again subjected to centrifugation. This operation was repeated three times to yield substantially homogenous hepatocytes. To the resultant hepatocytes was added 20 ml of a DMEM culture medium, and the cells were suspended therein to prepare a hepatocytes suspension.

Next, 900 ml of distilled water was added to 14.12 g of a Waymouth MB 752/1 culture medium (containing L-glutamine but containing no NaHCO₃) (manufactured by GIBCO). To this were added 2.24 g of NaHCO₃, 10 ml of an amphotericin B solution (ICN), and 10 ml of a penicillin streptomycin solution (manufactured by GIBCO), and this solution was stirred. This was adjusted into a pH of 7.4, and then the total amount thereof was set to 1000 ml. The resultant was filtrated with a 0.22 μm membrane filter and sterilized to prepare a Waymouth MB 752/1 culture medium solution.

The previously-prepared hepatocytes suspension was suspended into the prepared Waymouth MB 752/1 culture medium solution, and further the resultant suspension was inoculated onto the above-mentioned cell culture patterning substrate, which had the cell adhesion portions and the cell adhesion-inhibiting portions. This substrate was allowed to stand still in an incubator to which 5% CO₂ was supplied at 37° C. for 24 hours to cause the hepatocytes to adhere onto the entire surface of the substrate. This substrate was washedwith PBS two times to remove non-adhering cells and dead cells. Thereafter, the culture medium solution was exchanged for a new culture medium solution. While the exchange of the culture medium solution was repeated, the cells were continued to be cultured for 48 hours. The cells were then observed with an optical microscope. As a result, it was found out that the cells adhered along the cell adhesion portions of the cell culture patterning substrate.

### Example 2

### Formation of a Cell Adhesion-Inhibiting Layer

Six grams of isopropyl alcohol, 2 g of toluene, 0.4 g of an organosilane, TSL8114 (manufactured by GE Toshiba Silicones), and 0.1 g of poly(2-methoxyethyl)acrylate were mixed, and the mixture was heated at 100° C. for 20 minutes while stirred. This solution was applied onto a glass substrate (thickness: 0.7 mm) subjected to alkali treatment in advance by spin coating, and the substrate was dried at 150° C. for 10 minutes to advance hydrolysis and polycondensation reaction, thereby forming, on the substrate, a layer (thickness: about 60 nm) made of an organopolysiloxane having fluoroalkyl groups. In this way, a patterning substrate having a cell adhesion-inhibiting layer was formed.

(Patterning of the Patterning Substrate)

A photomask on which a photocatalyst layer was formed was put onto the patterning substrate to dispose the photocatalyst layer and the cell adhesion-inhibiting layer of the patterning substrate to face to each other, and then ultraviolet rays were irradiated from a mercury lamp through the photomask onto this patterning substrate at 5 J/cm² (measured wavelength: 254 nm) to yield a cell culture patterning substrate having a cell adhesive surface patterned so as to have unexposed portions having cell adhesion-inhibiting properties and exposed portions having cell adhesive properties.

(Cell Adhesion Process)

Experiment was held in the same way as in Example 1 and it was found out that the cells adhered along the cell adhesion portions of the cell culture patterning substrate.

### Example 3

A hole of 14 mm diameter was made at the center of the bottom face of a commercially available plastic dish (manufactured by Corning Inc.) of 35 mm diameter. Subsequently, a glass substrate of about 0.1 mm thickness was used to form a cell culture patterning substrate in the same way as in Example 1. This cell culture patterning substrate was cut into a 21 mm square. Thereafter, the glass substrate of the cut cell culture patterning substrate was stuck onto the above-mentioned plastic dish through an adhesive agent, KE45T (Shin-Etsu Chemical Co., Ltd).

(Adhesion of Cells)

The plastic dish was sterilized with 70% ethanol, and washed with PBS. Thereafter, the dish was washed with a DMEM culture medium. Cells were then cultured in the same way as in Example 1. As a result, it was found out that the cells adhered along the cell adhesion portions in the plastic dish.

### Example 4

A glass substrate about 0.1 mm in thickness was used to form a cell culture patterning substrate having a cell adhesion-inhibiting layer in the same way as in Example 2. This cell culture patterning substrate was cut into a 21 mm square. Thereafter, the glass substrate of the cut cell culture patterning substrate was stuck onto the above-mentioned plastic dish in the same way as in Example 3.

(Culture of Cells)

The cells were cultured in the plastic dish in the same way as in Example 3. As a result, it was found out that the cells adhered along the cell adhesion portions in the plastic dish.

### Example 5

(Formation of a Patterning Substrate)

Three grams of isopropyl alcohol, 0.2 g of an organosilane, TSL8114 (manufactured by GE Toshiba Silicones), and 0.2 g of a PEG-silane (Methoxypolyethylene glycol 5,000 trimethylsilyl ether, Fluka) were mixed, and the mixture was heated at 100° C. for 20 minutes while stirred. This solution was applied onto a washed glass substrate (thickness: 0.7 mm) by spin coating, and subsequently the substrate was subjected to heating treatment at 150° C. for 10 minutes, thereby yielding a patterning substrate wherein a cell adhesion-inhibiting layer (thickness: 160 nm) made of an organopolysiloxane layer containing polyethylene glycol was formed on the substrate.

(Patterning of the Patterning Substrate, and Adhesion of Cells)

The patterning substrate was patterned in the same way as in Example 1, so as to form a cell culture patterning substrate. Thereafter, cells were caused to adhere onto the cell culture patterning substrate in the same way as in Example 1. As a result, in the present examples also, it was found out that the cells adhered along the cell adhesion portions on the cell culture patterning substrate.

### Example 6

A glass substrate about 0.1 mm in thickness was used to form a cell culture patterning substrate having a cell adhesion-inhibiting layer in the same way as in Example 5. This cell culture patterning substrate was cut into a 21 mm square. Thereafter, the glass substrate of the cut cell culture patterning substrate was stuck onto the above-mentioned plastic dish in the same way as in Example 3.

(Culture of Cells)

Cells were cultured in the plastic dish in the same way as in Example 3. As a result, it was found out that cells adhered along the cell adhesion portions inside the plastic dish.

What is claimed is:

1. A patterning substrate, comprising: a base material; and a cell adhesion-inhibiting layer which is formed on the base material and comprises a cell adhesion-inhibiting material that has cell adhesion-inhibiting properties of inhibiting adhesion to cells and is denatured by action of a photocatalyst on a basis of irradiation with energy.

2. A patterning substrate, comprising: a base material; and a cell adhesion-inhibiting layer which is formed on the base material and comprises a binder and a cell adhesion-inhibiting material that has cell adhesion-inhibiting properties of inhibiting adhesion to cells and is decomposed or denatured by action of a photocatalyst on a basis of irradiation with energy.

3. The patterning substrate according to claim 1, wherein the cell adhesion-inhibiting layer comprises a cell adhesive material having cell adhesive properties at least after the material is irradiated with the energy.

4. The patterning substrate according to claim 2, wherein the cell adhesion-inhibiting layer comprises a cell adhesive material having cell adhesive properties at least after the material is irradiated with the energy.

5. The patterning substrate according to claim 1, wherein a light-shielding portion is formed on the base material.

6. The patterning substrate according to claim 2, wherein a light-shielding portion is formed on the base material.

7. A cell culture patterning substrate, wherein the cell adhesion-inhibiting layer of the patterning substrate according to claim 1 has a cell adhesion portion wherein the cell adhesion-inhibiting material is decomposed or denatured in a pattern form and a cell adhesion-inhibiting portion which is a region other than the cell adhesion portion.

8. A cell culture patterning substrate, wherein the cell adhesion-inhibiting layer of the patterning substrate according to claim 2 has a cell adhesion portion wherein the cell adhesion-inhibiting material is decomposed or denatured in a pattern form and a cell adhesion-inhibiting portion which is a region other than the cell adhesion portion.

9. A cell culture substrate, wherein the cells adhere onto the cell adhesion portion of the cell culture patterning substrate according to claim 7.

10. A cell culture substrate, wherein the cells adhere onto the cell adhesion of the cell culture patterning substrate according to claim 8.

11. A coating liquid for patterning substrate, comprising: a cell adhesion-inhibiting material that has cell adhesion-inhibiting properties of inhibiting adhesion to cells and is decomposed or denatured by action of a photocatalyst on a basis of irradiation with energy; and a cell adhesive material that has cell adhesive properties at least after the material is irradiated with the energy.

12. A method for producing a cell culture patterning substrate, comprising: a cell adhesion-inhibiting layer forming process of forming, on a base material, a cell adhesion-inhibiting layer comprising a cell adhesion-inhibiting material that has cell adhesion-inhibiting properties of inhibiting adhesion to cells and is decomposed or denatured by action of a photocatalyst on a basis of irradiation with energy; and
an energy irradiating process of arranging the cell adhesion-inhibiting layer and a photocatalyst-containing layer side substrate having a photocatalyst-containing layer comprising the photocatalyst and a base body to dispose the cell adhesion-inhibiting layer and the photocatalyst-containing layer facing each other, and then irradiating the energy onto a resultant from a given direction to form a pattern composed of a cell adhesion portion wherein the cell adhesion-inhibiting material comprised in the cell adhesion-inhibiting layer is decomposed or denatured, and a cell adhesion-inhibiting portion which is other than the cell adhesion portion.

13. A method for producing a cell culture substrate, comprising a cell adhesion process of causing the cells to adhere onto the cell adhesion portion of the cell culture patterning substrate produced by the method for producing the cell culture patterning substrate according to claim 12.
